(19) United States
(12) Patent Application Publication
(10) Pub. No.: US 2024/0157032 A1
(43) Pub. Date: May 16, 2024

(54) HIGH-STRENGTH MEDICAL FIBER COMPOSITE MATERIAL

(71) Applicant: CHANGZHOU INSTITUTE OF TECHNOLOGY, Changzhou (CN)

(72) Inventors: Che ZHAO, Changzhou (CN); Songxue LIU, Changzhou (CN); Chun FENG, Changzhou (CN); Zhiwei WU, Changzhou (CN); Yiwei ZHANG, Changzhou (CN); Wenbiao JIANG, Changzhou (CN); Xiaozhen LI, Changzhou (CN)

(73) Assignee: CHANGZHOU INSTITUTE OF TECHNOLOGY, Changzhou (CN)

(21) Appl. No.: 17/781,006

(22) PCT Filed: Nov. 29, 2021
(86) PCT No.: PCT/CN2021/133958

§ 371 Date: May 30, 2022

(30) Foreign Application Priority Data
Mar. 15, 2021 (CN) 202110275333.5

(51) Int. Cl. A61L 31/12; A61L 31/04; A61L 31/14

(52) U.S. Cl. CPC A61L 31/125; A61L 31/048; A61L 31/145

(57) ABSTRACT

A high-strength medical fiber composite material includes a sodium alginate hydrogel matrix and a fiber framework. The fiber framework is completely embedded in the sodium alginate hydrogel matrix and formed by compounding supporting layer fibers and reinforcing layer fibers. The reinforcing layer fibers are located above the supporting layer fibers. The reinforcing layer fibers and the supporting layer fibers are orthogonal to each other. According to the high-strength medical fiber composite material prepared in the present invention, the stiffness is improved by 3-4 orders of magnitude, the tensile strength is improved by 2-3 orders of magnitude, and the high-strength medical fiber composite material has high biocompatibility and safety and a great application prospect.

### CROSS REFERENCE TO THE RELATED APPLICATIONS

This application is the national phase entry of International Application No. PCT/CN2021/133958, filed on Nov. 29, 2021, which is based upon and claims priority to Chinese Patent Application No. 202110275333.5, filed on Mar. 15, 2021, the entire contents of which are incorporated herein by reference.

### TECHNICAL FIELD

The present invention relates to the technical field of medical composite materials, and in particular to a high-strength medical fiber composite material.

### BACKGROUND

As a three-dimensional network macromolecular polymer made from natural raw materials, a natural hydrogel can absorb a large amount of water and make the water bound in a three-dimensional network structure of the hydrogel. The natural hydrogel has high water absorption and moisture retention properties. A sodium alginate hydrogel has high biocompatibility and low toxicity and has been widely used in the fields including food, medicine and biomedicine. However, compared with synthetic macromolecular polymers, the sodium alginate hydrogel has poor mechanical performance (low stiffness and strength) and is likely to be broken under an external force. Therefore, when the sodium alginate hydrogel is separately used, practical application requirements are often difficult to meet. The sodium alginate hydrogel can only be used in dressings, absorbent fillers, drug carriers and other products with very low requirements on mechanical performance.

With development of medical technologies, clinical demands for artificial skins, artificial muscles, artificial tendons and other tissue repair materials are increasing. Therefore, how to maintain excellent biocompatibility and water absorption and moisture retention properties and significantly improve the mechanical performance of the sodium alginate hydrogel for higher practicality is a key problem to be solved urgently.

At present, the mechanical performance of the natural hydrogel is generally improved by scientific researchers by using a polymer compounding method. That is, two polymers are combined by using an interpenetrating network technology to achieve complementary and synergistic effects, so as to overcome defects of a single hydrogel. For example, according to a Chinese invention patent CN 104311841 A, a preparation method of a high-strength covalent/ion interpenetrating network easy-to-shape gel is disclosed, and it is further disclosed that the tensile strength of the prepared material can reach 1.8 MPa. According to the interpenetrating network technology, a three-dimensional network structure of a macromolecular polymer is merely improved at a molecular level. The overall mechanical performance, especially the strength or the stiffness, of the material is improved at a limited level, and is generally not improved by more than 2 orders of magnitude. Demands for load capacities of the artificial muscles and the artificial tendons still cannot be met.

Therefore, it is quite necessary to obtain a method for improving the stiffness and strength of the sodium alginate hydrogel by 2-3 orders of magnitude.

### SUMMARY

In view of the problems of the prior art, the present invention needs to solve the following technical problems. Due to poor mechanical performance and low stiffness and strength, a sodium alginate hydrogel is likely to be broken under an external force. Therefore, when the sodium alginate hydrogel is separately used, practical application requirements are often difficult to meet.

The present invention adopts the following technical solutions to solve the technical problems. The present invention provides a high-strength medical fiber composite material. The high-strength medical fiber composite material includes a sodium alginate hydrogel and a fiber framework. The fiber framework is completely embedded in the sodium alginate hydrogel, and a surface of the fiber framework is grafted with chemical anchor points with chemical bonding to the sodium alginate hydrogel.

Specifically, the fiber framework is formed by compounding several supporting layer fibers and several reinforcing layer fibers. The reinforcing layer fibers are located above the supporting layer fibers, and the reinforcing layer fibers and the supporting layer fibers are orthogonal to each other.

Specifically, the several reinforcing layer fibers are arranged at equal intervals. The several supporting layer fibers are arranged at equal intervals.

Specifically, every two adjacent reinforcing layer fibers are arranged at an interval of 0.4-0.8 mm. Every two adjacent supporting layer fibers are arranged at an interval of 1.2-1.6 mm.

Specifically, the reinforcing layer fibers are polyester fibers, nylon fibers or polyether ether ketone resin fibers with a diameter of 0.1-0.3 mm.

Specifically, the supporting layer fibers are polyester fibers, nylon fibers or polyether ether ketone resin fibers with a diameter of 0.1-0.3 mm.

Specifically, the chemical anchor points are amino silane functional groups obtained after the surface of the fiber framework is soaked in a surface treatment solution.

Specifically, the surface treatment solution is obtained by dissolving 1 g of sodium alginate, 241 mg of Solfo-NHS and 178 mg of EDC in 100 mL of an MES hydrate.

Specifically, the sodium alginate has a molecular weight of 26,000-28,000 Da.

Specifically, a preparation method of a high-strength medical fiber composite material includes the following steps:
(1) heating reinforcing layer fibers and supporting layer fibers for softening, stretching the fibers to reach a diameter of 0.1-0.3 mm, and cooling the obtained fibers to obtain fibers for use;
(2) conducting plasma etching on the fibers for use obtained in step (1) with oxygen as an etching gas at an etching powder of 30 W-40 W for 5 min-10 min by using a plasma cleaning machine;
(3) after the plasma etching, immediately soaking the fibers in a KH550 aqueous solution with a mass concentration of 2.5% for silanization for 3 h, cleaning the reinforcing layer fibers and the supporting layer fibers with deionized water and ethanol, and drying the fibers for later use;
(4) orienting, fixing and locking the reinforcing layer fibers and the supporting layer fibers treated in step (3) by using a combined mold to obtain a fiber framework where the reinforcing layer fibers and the supporting layer fibers are orthogonally compounded;
(5) soaking the fiber framework obtained in step (4) and the combined mold in a surface treatment solution for 18-24 h to make a surface of the fiber framework chemically grafted with chemical anchor points, taking out the fiber framework and the combined mold, cleaning the fiber framework and the combined mold with deionized water, and drying the fiber framework and the combined mold for later use;
(6) preparing sodium alginate into a sodium alginate aqueous solution with a mass fraction of 3-4%, heating the solution to 50-60° C. for uniform stirring, conducting ultrasonic treatment under 60 kHz until the solution is clear, and conducting standing for 12 h to obtain a sodium alginate hydrogel;
(7) injecting the sodium alginate hydrogel obtained in step (6) into a mold 2 of the combined mold dried in step (5), repeatedly smearing the sodium alginate hydrogel flat along an upper surface of the mold 2 by using a scraper to make the sodium alginate hydrogel in full contact with the fiber framework, and after the smearing is completed, conducting standing for 12 h to obtain a preformed material; and
(8) soaking the preformed material obtained in step (7) and the combined mold in a calcium chloride aqueous solution with a mass fraction of 1-3% for curing for 4 h, repeatedly cleaning the material with deionized water to remove residual calcium chloride on a surface of the material and drying the material, cutting the cured material in the combined mold according to a required size, and removing the material from the mold to obtain the high-strength medical fiber composite material.

The present invention has the following beneficial effects.
(1) The medical fiber reinforced composite material provided in the present invention is obtained by embedding a fiber framework consisting of high-strength medical fibers such as nylon, polyester and polyether ketone resin in a sodium alginate hydrogel matrix. The overall mechanical performance of the medical composite material is effectively improved. The stiffness can be improved by 3-4 orders of magnitude, and the tensile strength can be improved by 2-3 orders of magnitude. The medical composite material has high biocompatibility and safety.
(2) The medical fiber reinforced composite material provided in the present invention has high designability and adjustability. The overall mechanical performance of the material can be regulated by adjusting the density of the reinforcing layer fibers based on specific application demands. When the intervals of the fibers are reduced and the fibers are tightly arranged, the overall stiffness and strength of the composite material are improved, and the flexibility is reduced. When the intervals of the fibers are increased and the fibers are sparsely arranged, the overall stiffness and strength of the material are reduced, and the flexibility is improved.
(3) According to the preparation method of a medical fiber reinforced composite material provided in the present invention, fiber orienting, weaving, density adjustment, fixing, locking, injection molding and other functions can be achieved by only requiring a simple mold structure. Complicated and expensive machines and equipment are not required. The method has the characteristics of low cost, high efficiency, simple operation and mass production.
(4) The method of the present invention has universality and can be applied to nylon, polyester, polyether ketone resin and other medical fibers with different physical and chemical properties.

### BRIEF DESCRIPTION OF THE DRAWINGS

FIG. 1 is a schematic diagram showing a preparation process of a high-strength medical fiber composite material in the present invention.

FIG. 2 is a schematic structure diagram of a high-strength medical fiber composite material in the present invention.

FIG. 3 is a schematic structure diagram of a mold 1.

FIG. 4 is a schematic structure diagram of a combined mold.

FIG. 5 is a schematic diagram showing a process of preparing a fiber framework by using the mold 1.

In the drawings: 1, supporting layer fiber; 2, reinforcing layer fiber; 3, chemical anchor point; 4, sodium alginate hydrogel; 5, mold 1; 6, mold 2; 7, fixing base plate; and 8, screw hole.

### DETAILED DESCRIPTION OF THE EMBODIMENTS

The present invention is further described in detail in conjunction with the accompanying drawings.

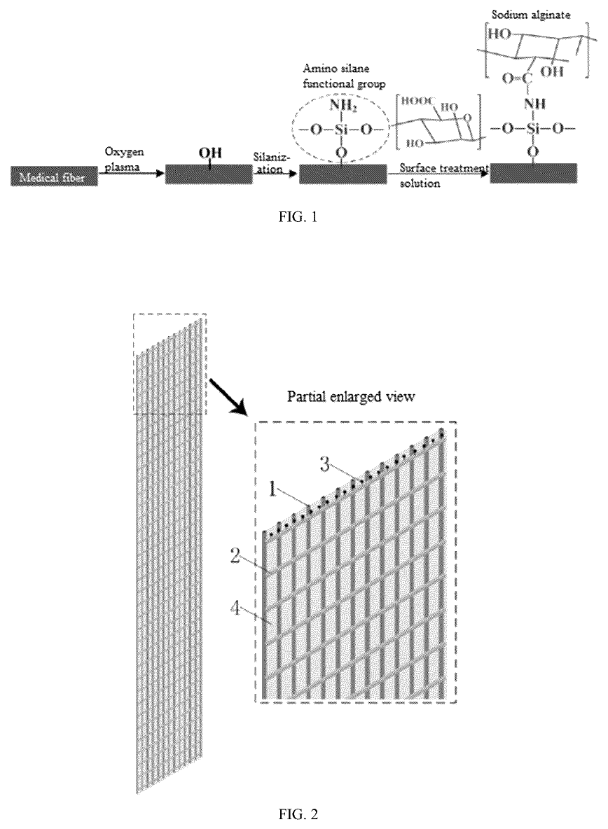

A preparation process of a high-strength medical fiber composite material in the following embodiments of the present invention is shown in FIG. 1. FIG. 2 is a schematic structure diagram of a high-strength medical fiber composite material prepared in Embodiments 1-7.

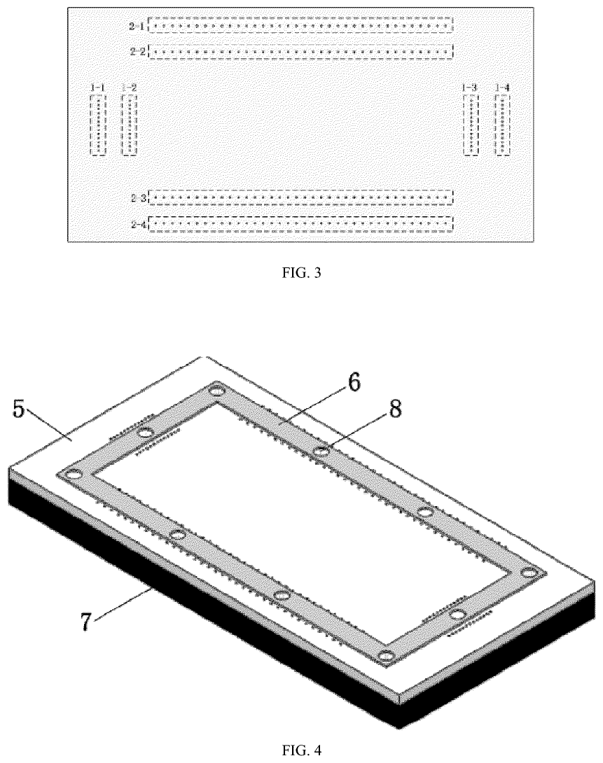

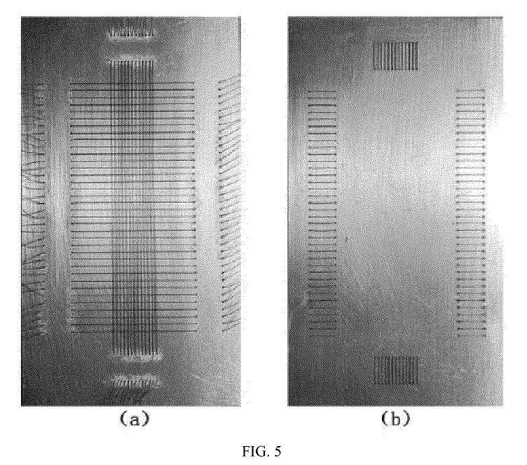

A combined mold used in the following embodiments and comparative examples of the present invention is shown in FIG. 4. The combined mold consists of a mold 1 and a mold 2. As shown in FIG. 3, the mold 1 is a stainless steel plate with two rows of equidistant circular holes formed in each side. The stainless steel plate has a thickness of 1-3 mm. Adjacent two rows of horizontal circular holes in the stainless steel plate have a distance of 6 mm. Adjacent two rows of longitudinal circular holes in the stainless steel plate have a distance of 5 mm. The hole has a diameter of 0.2-0.4 mm. Any row of horizontal circular holes in the stainless steel plate have a center-to-center distance of 1.2-1.6 mm. Any row of longitudinal circular holes in the stainless steel plate have a center-to-center distance of 0.4-0.8 mm. The stainless steel plate is provided with 2-1, 2-2, 2-3 and 2-4 in the horizontal direction and 1-1, 1-2, 1-3 and 1-4 in the longitudinal direction, the circular holes of 1-2 correspond to the circular holes of 1-3 one to one, and the circular holes of 2-2 correspond to the circular holes of 2-3 one to one. The mold 2 is prepared from a Teflon material and has a thickness of 0.25-0.5 mm, an inner frame length×an inner frame width=29 mm×68 mm, and a frame width of 3-4 mm. During use, supporting layer fibers are sequentially penetrated through the one-to-one corresponding circular holes formed in 2-1, 2-2, 2-3 and 2-4 in the mold 1 and then locked. Reinforcing layer fibers are sequentially penetrated through the one-to-one corresponding circular holes formed in 1-1, 1-2, 1-3 and 1-4 in the mold 1 and then locked. Then, four frames of the mold 2 exactly fall in a non-porous area between the two rows of corresponding circular holes in every side of the mold 1. The mold 1 and the mold 2 are fixed to a base plate together by using a threaded connection method (a fiber framework prepared on the combined mold is shown in FIG. 5). Next, a sodium alginate hydrogel is injected into the mold 2, and subsequent operations are conducted.

A surface treatment solution used in the following embodiments and comparative examples of the present invention is obtained by dissolving 1 g of sodium alginate (with a molecular weight of 26,000-28,000 Da), 241 mg of N-hydroxythiosuccinimide (Solfo-NHS, CAS: 106627-54-7) and 178 mg of 1-ethyl-(3-dimethylaminopropyl)carbodiimide (EDC, CAS: 25952-53-8) in 100 mL of a morpholinoethanesulfonic acid hydrate (MES hydrate, CAS: 1266615-59-1).

The sodium alginate used in the following embodiments and comparative examples of the present invention has a molecular weight of 26,000-28,000 Da.

### Embodiment 1

(1) Polyester fibers and polyether ether ketone resin fibers were heated for softening, stretched and then cooled to obtain fibers with a length of 100 mm, the polyester fibers had a diameter of 0.12±0.01 mm, and the polyether ether ketone resin fibers had a diameter of 0.16±0.01 mm.
(2) Plasma etching was conducted on the polyester fibers and the polyether ether ketone resin fibers obtained in step (1) with oxygen as an etching gas by using a plasma cleaning machine, the polyester fibers were etched at an etching powder of 30 W for 5 min, and the polyether ether ketone resin fibers were etched at an etching powder of 40 W for 10 min.
(3) After the plasma etching, the polyester fibers and the polyether ether ketone resin fibers were immediately soaked in a KH550 aqueous solution with a mass concentration of 2.5% for silanization for 3 h, cleaned with deionized water and ethanol and then dried for later use.
(4) The 9 polyester fibers treated in step (3) were sequentially penetrated through the one-to-one corresponding circular holes formed in 1-1, 1-2, 1-3 and 1-4 in the mold 1 and then locked, and the 36 polyether ether ketone resin fibers treated in step (3) were sequentially penetrated through the one-to-one corresponding circular holes formed in 2-1, 2-2, 2-3 and 2-4 in the mold 1 and then locked to obtain a fiber framework where the polyester fibers and the polyether ether ketone resin fibers were orthogonally compounded.
(5) The fiber framework obtained in step (4) and the combined mold were soaked in the surface treatment solution for 20 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 2

(1) Nylon fibers were heated for softening, stretched and then cooled to obtain fibers for use with a length of 100 mm, 9 fibers had a diameter of 0.12±0.01 mm, and 36 fibers had a diameter of 0.16±0.01 mm.
(2) Plasma etching was conducted on the nylon fibers obtained in step (1) with oxygen as an etching gas at an etching powder of 35 W for 7 min by using a plasma cleaning machine.
(3) After the plasma etching, the nylon fibers were immediately soaked in a KH550 aqueous solution with a mass concentration of 2.5% for silanization for 3 h, cleaned with deionized water and ethanol and then dried for later use.
(4) The 9 nylon fibers (with a diameter of 0.12±0.01 mm) treated in step (3) were sequentially penetrated through the one-to-one corresponding circular holes formed in 1-1, 1-2, 1-3 and 1-4 in the mold 1 and then locked, and the 36 nylon fibers (with a diameter of 0.16±0.01 mm) were sequentially penetrated through the one-to-one corresponding circular holes formed in 2-1, 2-2, 2-3 and 2-4 in the mold 1 and then locked to obtain a fiber framework where the nylon fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 4%, the solution was heated to 50° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 3% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 3

(1) Nylon fibers and polyether ether ketone resin fibers were heated for softening, stretched and then cooled to obtain fibers for use with a length of 100 mm, the nylon fibers had a diameter of 0.16±0.01 mm, and the polyether ether ketone resin fibers had a diameter of 0.12±0.01 mm.
(2) Plasma etching was conducted on the nylon fibers and the polyether ether ketone resin fibers obtained in step (1) with oxygen as an etching gas by using a plasma cleaning machine, the nylon fibers were etched at an etching powder of 35 W for 7 min, and the polyether ether ketone resin fibers were etched at an etching powder of 40 W for 10 min.
(3) After the plasma etching, the nylon fibers and the polyether ether ketone resin fibers were immediately soaked in a KH550 aqueous solution with a mass concentration of 2.5% for silanization for 3 h, cleaned with deionized water and ethanol and then dried for later use.
(4) The 17 nylon fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) treated in step (3) were sequentially penetrated through the one-to-one corresponding circular holes formed in 2-1, 2-2, 2-3 and 2-4 in the mold 1 and then locked to obtain a fiber framework where the nylon fibers and the polyether ether ketone resin fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 1% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 4

(1) Polyether ether ketone resin fibers were heated for softening, stretched and then cooled to obtain fibers for use with a length of 100 mm, 17 fibers had a diameter of 0.16±0.01 mm, and 36 fibers had a diameter of 0.12±0.01 mm.
(2) Plasma etching was conducted on the polyether ether ketone resin fibers obtained in step (1) with oxygen as an etching gas at an etching powder of 40 W for 10 min by using a plasma cleaning machine.
(3) After the plasma etching, the polyether ether ketone resin fibers were immediately soaked in a KH550 aqueous solution with a mass concentration of 2.5% for silanization for 3 h, cleaned with deionized water and ethanol and then dried for later use.
(4) The 17 polyether ether ketone resin fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) were sequentially penetrated through the one-to-one corresponding circular holes formed in 2-1, 2-2, 2-3 and 2-4 in the mold 1 and then locked to obtain a fiber framework where the polyether ether ketone resin fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 5

(1) Nylon fibers and polyether ether ketone resin fibers were heated for softening, stretched and then cooled to obtain fibers for use with a length of 100 mm, the nylon fibers had a diameter of 0.16±0.01 mm, and the polyether ether ketone resin fibers had a diameter of 0.12±0.01 mm.
(2) Plasma etching was conducted on the nylon fibers and the polyether ether ketone resin fibers obtained in step (1) with oxygen as an etching gas by using a plasma cleaning machine, the nylon fibers were etched at an etching powder of 35 W for 7 min, and the polyether ether ketone resin fibers were etched at an etching powder of 40 W for 10 min.
(3) After the plasma etching, the nylon fibers and the polyether ether ketone resin fibers were immediately soaked in a KH550 aqueous solution with a mass concentration of 2.5% for silanization for 3 h, cleaned with deionized water and ethanol and then dried for later use.
(4) The 9 nylon fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) treated in step (3) were sequentially penetrated through the one-to-one corresponding circular holes formed in 2-1, 2-2, 2-3 and 2-4 in the mold 1 and then locked to obtain a fiber framework where the nylon fibers and the polyether ether ketone resin fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 6

(1) Polyester fibers were heated for softening, stretched and then cooled to obtain fibers for use with a length of 100 mm, 9 fibers had a diameter of 0.16±0.01 mm, and 36 fibers had a diameter of 0.12±0.01 mm.
(2) Plasma etching was conducted on the polyester fibers obtained in step (1) with oxygen as an etching gas at an etching powder of 30 W for 5 min by using a plasma cleaning machine.
(3) After the plasma etching, the polyester fibers were immediately soaked in a KH550 aqueous solution with a mass concentration of 2.5% for silanization for 3 h, cleaned with deionized water and ethanol and then dried for later use.
(4) The 9 polyester fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 polyester fibers (with a diameter of 0.12±0.01 mm) treated in step (3) were sequentially penetrated through the one-to-one corresponding circular holes formed in 2-1, 2-2, 2-3 and 2-4 in the mold 1 and then locked to obtain a fiber framework where the polyester fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

### Embodiment 7

(1) Polyester fibers and nylon fibers were heated for softening, stretched and then cooled to obtain fibers for use with a length of 100 mm, the polyester fibers had a diameter of 0.16±0.01 mm, and the nylon fibers had a diameter of 0.12±0.01 mm.
(2) Plasma etching was conducted on the polyester fibers and the nylon fibers obtained in step (1) with oxygen as an etching gas by using a plasma cleaning machine, and the polyester fibers were etched at an etching powder of 30 W for 5 min.
(3) After the plasma etching, the polyester fibers and the nylon fibers were immediately soaked in a KH550 aqueous solution with a mass concentration of 2.5% for silanization for 3 h, cleaned with deionized water and ethanol and then dried for later use.
(4) The 17 polyester fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 nylon fibers (with a diameter of 0.12±0.01 mm) treated in step (3) were sequentially penetrated through the one-to-one corresponding circular holes formed in 2-1, 2-2, 2-3 and 2-4 in the mold 1 and then locked to obtain a fiber framework where the polyester fibers and the nylon fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

Comparative Example 1 was almost the same as Embodiment 4, but had the following differences.
(1) Polyether ether ketone resin fibers were heated for softening, stretched and then cooled to obtain fibers for use with a length of 100 mm, 17 fibers had a diameter of 0.16±0.01 mm, and 36 fibers had a diameter of 0.12±0.01 mm.
(2) The polyether ether ketone resin fibers were soaked in a KH550 aqueous solution with a mass concentration of 2.5% for silanization for 3 h, cleaned with deionized water and ethanol and then dried for later use.
(3) The 17 polyether ether ketone resin fibers (with a diameter of 0.16±0.01 mm) treated in step (2) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) were sequentially penetrated through the one-to-one corresponding circular holes formed in 2-1, 2-2, 2-3 and 2-4 in the mold 1 and then locked to obtain a fiber framework where the polyether ether ketone resin fibers were orthogonally compounded.
(4) The fiber framework obtained in step (3) and the combined mold were soaked in the surface treatment solution for 24 h to make a surface of the fiber framework chemically grafted with chemical anchor points, and the fiber framework and the combined mold were taken out, cleaned with deionized water and then dried for later use.
(5) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(6) The sodium alginate hydrogel obtained in step (5) was injected into the mold 2 of the combined mold dried in step (4) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(7) The preformed material obtained in step (6) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

Comparative Example 2 was almost the same as Embodiment 4, but had the following differences.
(1) Polyether ether ketone resin fibers were heated for softening, stretched and then cooled to obtain fibers for use with a length of 100 mm, 17 fibers had a diameter of 0.16±0.01 mm, and 36 fibers had a diameter of 0.12±0.01 mm.
(2) Plasma etching was conducted on the polyether ether ketone resin fibers obtained in step (1) with oxygen as an etching gas at an etching powder of 40 W for 10 min by using a plasma cleaning machine.
(3) The 17 polyether ether ketone resin fibers (with a diameter of 0.16±0.01 mm) treated in step (2) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) were sequentially penetrated through the one-to-one corresponding circular holes formed in 2-1, 2-2, 2-3 and 2-4 in the mold 1 and then locked to obtain a fiber framework where the polyether ether ketone resin fibers were orthogonally compounded.
(4) The fiber framework obtained in step (3) and the combined mold were soaked in the surface treatment solution for 24 h to make a surface of the fiber framework chemically grafted with chemical anchor points, and the fiber framework and the combined mold were taken out, cleaned with deionized water and then dried for later use.
(5) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(6) The sodium alginate hydrogel obtained in step (5) was injected into the mold 2 of the combined mold dried in step (4) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(7) The preformed material obtained in step (6) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

Comparative Example 3 was almost the same as Embodiment 4, but had the following differences.
(1) Polyether ether ketone resin fibers were heated for softening, stretched and then cooled to obtain fibers for use with a length of 100 mm, 17 fibers had a diameter of 0.16±0.01 mm, and 36 fibers had a diameter of 0.12±0.01 mm.
(2) Plasma etching was conducted on the polyether ether ketone resin fibers obtained in step (1) with oxygen as an etching gas at an etching powder of 40 W for 10 min by using a plasma cleaning machine.
(3) After the plasma etching, the polyether ether ketone resin fibers were immediately soaked in a KH550 aqueous solution with a mass concentration of 2.5% for silanization for 3 h, cleaned with deionized water and ethanol and then dried for later use.
(4) The 17 polyether ether ketone resin fibers (with a diameter of 0.16±0.01 mm) treated in step (3) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) were sequentially penetrated through the one-to-one corresponding circular holes formed in 2-1, 2-2, 2-3 and 2-4 in the mold 1 and then locked to obtain a fiber framework where the polyether ether ketone resin fibers were orthogonally compounded.
(5) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(6) The sodium alginate hydrogel obtained in step (5) was injected into the mold 2 of the dried combined mold and repeatedly smeared flat along an upper surface of the mold 2 by using a scraper to make the sodium alginate hydrogel in full contact with the fiber framework, and after the smearing was completed, standing was conducted for 12 h to obtain a preformed material.
(7) The preformed material obtained in step (6) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

Comparative Example 4 was almost the same as Embodiment 5, but had the following differences.
(1) Nylon fibers and polyether ether ketone resin fibers were heated for softening, stretched and then cooled to obtain fibers for use with a length of 100 mm, the nylon fibers had a diameter of 0.08±0.01 mm, and the polyether ether ketone resin fibers had a diameter of 0.12±0.01 mm.
(2) Plasma etching was conducted on the nylon fibers and the polyether ether ketone resin fibers obtained in step (1) with oxygen as an etching gas by using a plasma cleaning machine, the nylon fibers were etched at an etching powder of 35 W for 7 min, and the polyether ether ketone resin fibers were etched at an etching powder of 40 W for 10 min.
(3) After the plasma etching, the nylon fibers and the polyether ether ketone resin fibers were immediately soaked in a KH550 aqueous solution with a mass concentration of 2.5% for silanization for 3 h, cleaned with deionized water and ethanol and then dried for later use.
(4) The 9 nylon fibers (with a diameter of 0.08±0.01 mm) treated in step (3) were sequentially penetrated through the one-to-one corresponding circular holes formed in 1-1, 1-2, 1-3 and 1-4 in the mold 1 and then locked, and the 36 polyether ether ketone resin fibers (with a diameter of 0.12±0.01 mm) treated in step (3) were sequentially penetrated through the one-to-one corresponding circular holes formed in 2-1, 2-2, 2-3 and 2-4 in the mold 1 and then locked to obtain a fiber framework where the nylon fibers and the polyether ether ketone resin fibers were orthogonally compounded.
(5) The fiber framework obtained in step (4) and the combined mold were soaked in the surface treatment solution for 24 h to make a surface of the fiber framework chemically grafted with chemical anchor points, and the fiber framework and the combined mold were taken out, cleaned with deionized water and then dried for later use.
(6) The sodium alginate was prepared into a sodium alginate aqueous solution with a mass fraction of 3.5%, the solution was heated to 60° C. for uniform stirring, ultrasonic treatment was conducted under 60 kHz until the solution was clear, and standing was conducted for 12 h to obtain a sodium alginate hydrogel.
(7) The sodium alginate hydrogel obtained in step (6) was injected into the mold 2 of the combined mold dried in step (5) and repeatedly smeared flat along an upper surface of the mold 2 by using a scraper to make the sodium alginate hydrogel in full contact with the fiber framework, and after the smearing was completed, standing was conducted for 12 h to obtain a preformed material.
(8) The preformed material obtained in step (7) and the combined mold were soaked in a calcium chloride aqueous solution with a mass fraction of 2% for curing for 4 h, the material was repeatedly cleaned with deionized water to remove residual calcium chloride on a surface of the material and dried, the cured material in the combined mold was cut according to a required size, and the material was removed from the mold to obtain a high-strength medical fiber composite material.

Mechanical Performance Test:

8 g of a sodium alginate aqueous solution with a mass fraction of 3.5% was spread on a frame type organic glass model plate and soaked in a 2% calcium chloride solution for complete curing to obtain a film with an average thickness of 0.3 mm. The film was cut into a blank sample with a length of 60 mm and a width of 9.8 mm.

According to methods in Embodiments 1-7 and Comparative Examples 1-4, samples with a thickness of 0.3 mm, a length of 60 mm and a width of 9.8 mm were separately prepared. In addition, it was ensured that surfaces of the samples were smooth and free of bubbles, cracks, layers, mechanical damages and other defects.

A tensile test was carried out on an Instron model 5544 universal material testing machine. The tensile elastic modulus and tensile strength of the samples were determined. The gauge length of a fixture was 40 mm, and the tensile speed was 0.5 mm/s. Each sample was tested in 3 parallel tests to obtain an average value, and test results were shown in the following Table 1.

TABLE 1

| | Tensile elastic | |
|---|---|---|
| Test item | modulus (MPa) | Tensile strength (MPa) |
| Blank sample | 0.072 | 0.016 |
| Embodiment 1 | 76.39 | 2.06 |
| Embodiment 2 | 81.26 | 2.18 |
| Embodiment 3 | 326.43 | 11.58 |
| Embodiment 4 | 443.57 | 14.16 |
| Embodiment 5 | 92.43 | 2.59 |
| Embodiment 6 | 86.39 | 2.23 |
| Embodiment 7 | 306.43 | 9.78 |
| Comparative Example 1 | 431.52 | 5.64 |
| Comparative Example 2 | 435.41 | 6.79 |
| Comparative Example 3 | 433.62 | 6.62 |
| Comparative Example 4 | 36.48 | 1.14 |

According to test results shown in Table 1, it could be seen that compared with a sodium alginate hydrogel monomer, the mechanical performance of the medical fiber reinforced composite material prepared by using the preparation method provided in the present invention was significantly improved. The tensile elastic modulus was improved by 3-4 orders of magnitude, and the breaking strength was improved by 2-3 orders of magnitude.

Cytotoxicity Test:

The cytotoxicity of the medical fiber reinforced composite materials in Embodiments 1-7 was determined by using an MTT colorimetric method. Mouse fibroblasts (L929) were selected in the test, and test results were shown in Table 2.

TABLE 2

| | Relative growth rate RGR | |
|---|---|---|
| Embodiment | (%) | Grade |
| 1 | 101.3 | 0 |
| 2 | 103.2 | 0 |
| 3 | 105.6 | 0 |
| 4 | 102.4 | 0 |
| 5 | 101.1 | 0 |
| 6 | 103.3 | 0 |
| 7 | 104.2 | 0 |

From Table 2, it could be seen that the medical fiber reinforced composite materials prepared in Embodiments 1-7 had low cytotoxicity on the mouse fibroblasts (L929), the relative growth rate of the cells was 100% or more, and the grade was grade 0 (best grade). It was indicated that the medical fiber reinforced composite material prepared in the present invention could be used as a biomedical material.

Inspired by the ideal embodiments of the present invention and based on the descriptions above, various changes and modifications can be made by related persons without departing from the scope of technical ideas of the present invention. The technical scope of the present invention is not limited to the descriptions in the specification and needs to be determined according to the scope of the claims.

What is claimed is:

1. A high-strength medical fiber composite material, comprising a sodium alginate hydrogel and a fiber framework, wherein the fiber framework is completely embedded in the sodium alginate hydrogel, and a surface of the fiber framework is grafted with chemical anchor points with chemical bonding to the sodium alginate hydrogel.

2. The high-strength medical fiber composite material according to claim 1, wherein the fiber framework is formed by compounding supporting layer fibers and reinforcing layer fibers, the reinforcing layer fibers are located above the supporting layer fibers, and the reinforcing layer fibers and the supporting layer fibers are orthogonal to each other.

3. The high-strength medical fiber composite material according to claim 2, wherein the reinforcing layer fibers are arranged at equal intervals, and the supporting layer fibers are arranged at equal intervals.

4. The high-strength medical fiber composite material according to claim 3, wherein every two adjacent reinforcing layer fibers are arranged at an interval of 0.4-0.8 mm, and every two adjacent supporting layer fibers are arranged at an interval of 1.2-1.6 mm.

5. The high-strength medical fiber composite material according to claim 2, wherein the reinforcing layer fibers are polyester fibers, nylon fibers, or polyether ether ketone resin fibers with a diameter of 0.1-0.3 mm.

6. The high-strength medical fiber composite material according to claim 2, wherein the supporting layer fibers are polyester fibers, nylon fibers, or polyether ether ketone resin fibers with a diameter of 0.1-0.3 mm.

7. The high-strength medical fiber composite material according to claim 1, wherein the chemical anchor points are amino silane functional groups obtained after the surface of the fiber framework is soaked in a surface treatment solution.

8. The high-strength medical fiber composite material according to claim 7, wherein the surface treatment solution is obtained by dissolving 1 g of sodium alginate, 241 mg of Solfo-NHS and 178 mg of EDC in 100 mL of an IVIES hydrate.

9. A preparation method of the high-strength medical fiber composite material according to claim 1, comprising the following steps:
1) heating reinforcing layer fibers and supporting layer fibers for softening, stretching the reinforcing layer fibers and the supporting layer fibers to each reach a diameter of 0.1-0.3 mm to obtain stretched fibers, and cooling the stretched fibers to obtain fibers for use;
2) conducting plasma etching on the fibers for use obtained in step 1) with oxygen as an etching gas by using a plasma cleaning machine to obtain etched fibers;
3) after the plasma etching, immediately soaking the etched fibers in a KH550 aqueous solution with a mass concentration of 2.5% for silanization for 3 h to obtain silanized reinforcing layer fibers and silanized support layer fibers, cleaning the silanized reinforcing layer fibers and the silanized supporting layer fibers with deionized water and ethanol, and drying the silanized reinforcing layer fibers and the silanized supporting layer fibers for later use;
4) orienting, fixing and locking the silanized reinforcing layer fibers and the silanized supporting layer fibers treated in step 3) by using a combined mold to obtain the fiber framework where the silanized reinforcing layer fibers and the silanized supporting layer fibers are orthogonally compounded;
5) soaking the fiber framework obtained in step 4) and the combined mold in a surface treatment solution to make the surface of the fiber framework chemically grafted with the chemical anchor points, taking out the fiber framework and the combined mold, cleaning the fiber framework and the combined mold with deionized water, and drying the fiber framework and the combined mold for later use;
6) preparing sodium alginate into a sodium alginate aqueous solution, heating the sodium alginate aqueous solution to 50-60° C. for uniform stirring, conducting ultrasonic treatment under 60 kHz until the sodium alginate aqueous solution is clear, and conducting standing for 12 h to obtain the sodium alginate hydrogel;
7) injecting the sodium alginate hydrogel obtained in step 6) into a mold of the combined mold dried in step 5), repeatedly smearing the sodium alginate hydrogel flat along an upper surface of the mold by using a scraper to make the sodium alginate hydrogel in full contact with the fiber framework, and after the smearing is completed, conducting standing for 12 h to obtain a preformed material; and
8) soaking the preformed material obtained in step 7) and the combined mold in a calcium chloride aqueous solution for curing for 4 h to obtain a cured material, repeatedly cleaning the cured material with deionized water to remove residual calcium chloride on a surface of the cured material and drying the cured material, cutting the cured material in the combined mold according to a required size, and removing the cured and cut material from the mold to obtain the high-strength medical fiber composite material.

10. The high-strength medical fiber composite material according to claim 3, wherein the reinforcing layer fibers are polyester fibers, nylon fibers, or polyether ether ketone resin fibers with a diameter of 0.1-0.3 mm.

11. The high-strength medical fiber composite material according to claim 4, wherein the reinforcing layer fibers are polyester fibers, nylon fibers, or polyether ether ketone resin fibers with a diameter of 0.1-0.3 mm.

12. The high-strength medical fiber composite material according to claim 3, wherein the supporting layer fibers are polyester fibers, nylon fibers, or polyether ether ketone resin fibers with a diameter of 0.1-0.3 mm.

13. The high-strength medical fiber composite material according to claim 4, wherein the supporting layer fibers are polyester fibers, nylon fibers, or polyether ether ketone resin fibers with a diameter of 0.1-0.3 mm.
